(19) United States
(12) Patent Application Publication
(10) Pub. No.: US 2009/0093726 A1
(43) Pub. Date: Apr. 9, 2009

(54) CARDIOVASCULAR ULTRASOUND PROBE AND ULTRASOUND IMAGE SYSTEM

(75) Inventors: Shuichi TAKAYAMA, Tokyo (JP); Toshiki TERAYAMA, Tokyo (JP); Takuya IMAHASHI, Kawasaki-shi (JP); Yoshinao OAKI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignees: OLYMPUS MEDICAL SYSTEMS CORP., Tokyo (JP); OLYMPUS CORPORATION, Tokyo (JP)

(21) Appl. No.: 12/244,459

(22) Filed: Oct. 2, 2008

Related U.S. Application Data
(60) Provisional application No. 60/997,704, filed on Oct. 4, 2007.

(51) Int. Cl. A61B 8/14

(52) U.S. Cl. 600/466

(57) ABSTRACT

An ultrasound image system includes: an ultrasound probe; a signal-transmitting-and-receiving section for transmitting signals to the ultrasound probe and receiving signals from the ultrasound probe; and an image-displaying section for displaying an ultrasound image obtained by means of the signal-transmitting-and-receiving section, wherein the ultrasound probe includes: a tip section which is provided with: a plurality of ultrasound-wave-transmitting sections for transmitting ultrasound waves to an tissue; and a plurality of ultrasound-wave-receiving sections for receiving signals reflected by the tissue and obtained by means of the ultrasound-wave-transmitting section; and a flexible catheter tube provided with: a first lumen capable of inserting an instrument for treating the tissue from a proximal end section of the ultrasound probe therethrough and exposing the instrument at a position facing the tip section; and a second lumen having an maneuvering wire inserted therethrough and being capable of moving the tip section.

### BACKGROUND OF THE INVENTION

1. Field of the Invention

The present invention relates to an ultrasound probe inserted into a heart or a blood vessel, and relates to an ultrasound image system, provided with the ultrasound probe, usable with various instruments incorporated therewith.

2. Background Art

Conventionally known instruments treat structural heart disease, e.g., patent foramen ovale, by inserting a distal end thereof into a blood vessel until reaching a heart. (See, e.g., U.S. Publication 2006/0271030) The instrument used therefor is moved to a treatment object position, e.g., an oval foramen, while observing a position of the distal end in an image obtained by a chest X-ray Fluoroscopy image.

However, it is difficult to recognize coordinates of the treatment object position in a depth direction based on an image since the chest X-ray Fluoroscopy image is a two-dimensional image. Therefore, an operator must check a plurality of X-ray Fluoroscopy images to permit the distal end of the instrument to reach to the treatment object position, and to fine-control the distal end in depth direction by trial and error. It is problematic because tissue at an unintended location may be damaged by the distal end of the instrument, or longer manipulation time may increase radiation dose to a patient.

In a conceivable method of moving an instrument while obtaining information with respect to the depth direction, many ultrasound probes currently used therefor are inserted through a cardiovascular vein and transmit ultrasound wave in a radial direction. A device under development for transmitting ultrasound wave in a forward direction has a defect because an ultrasound transducer is so small that it cannot provide significant resolution or sufficient observation distance.

Also, the user maneuvering both the instrument and the ultrasound probe must endure a complex operation. In addition, information obtained by the ultrasound probe is difficult to be used effectively for delivering the instrument, since the instrument may sometimes move out of an image obtained by the ultrasound probe because both components are operable separately.

The present invention was conceived in consideration of the aforementioned circumstances, and an object thereof is to provide a cardiovascular ultrasound probe that can obtain ultrasound images while enabling in-sight observation of the distal end of the inserted instrument.

From a different point of view, another object of the present invention is to provide a cardiovascular ultrasound probe that can obtain ultrasound images while continuously enabling in-sight observation of the distal end of an instrument by providing an ultrasound observation apparatus using an ultrasound probe, inserted through the inserted instrument to a heart or a blood vessel, capable of diagnosis with respect to an object observed in a forward direction.

From further different point of view, another object of the present invention is to obtain ultrasound images of a heart or a blood vessel therein with a significant observation distance.

Yet further different object of the present invention is to provide an ultrasound image system capable of moving a distal end thereof and conducting treatment while guiding the distal end of an instrument reliably in accordance with information obtained based on ultrasound images.

### BRIEF DESCRIPTION OF DRAWINGS

FIG. 1 is a schematic view of an ultrasound image system according to a first embodiment of the present invention.

FIG. 2 is an enlarged view showing a part of a distal end section of the ultrasound image system in cross-section.

FIG. 3 shows locations with respect to a heart and a patent foramen ovale.

FIG. 4 is a flowchart showing a sequence of treatments for the patent foramen ovale by using the ultrasound image system.

FIG. 5 illustrates a step of treatments with respect to the patent foramen ovale.

FIG. 6 illustrates a step of the treatments.

FIG. 7 illustrates the movement of the distal end section of the ultrasound image system used in the aforementioned treatments.

FIGS. 8A and 8B show an ultrasound image of the ultrasound image system used in the aforementioned treatments.

FIG. 9 illustrates a step of treatments with respect to the patent foramen ovale.

FIG. 10 illustrates a step of the treatments.

FIG. 11 is an enlarged schematic view showing a part of the distal end section of an ultrasound image system according to a second embodiment of the present invention.

FIG. 12 is a flowchart showing a sequence of treatments for a chronic total occlusion in an arteria coronaria by using the ultrasound image system.

FIG. 13 illustrates a step of the treatments with respect to the chronic total occlusion.

FIG. 14 illustrates a step of the treatments.

FIG. 15 illustrates a step of the treatments.

FIG. 16 illustrates an ultrasound image obtained by the ultrasound image system used in the aforementioned treatments.

FIG. 17 illustrates an ultrasound image obtained by the ultrasound image system used in the aforementioned treatments.

FIG. 18 illustrates an ultrasound image obtained by the ultrasound image system used in the aforementioned treatments.

FIG. 19 illustrates an ultrasound image obtained by the ultrasound image system used in the aforementioned treatments.

FIG. 20 is an enlarged schematic view showing a part of the distal end section of an ultrasound image system according to a third embodiment of the present invention.

FIG. 21 illustrates a preliminary treatment conducted prior to catheter ablation for atrial fibrillation by using the ultrasound image system.

FIG. 22 illustrates a step of the catheter ablation.

FIG. 23 illustrates the movement of the distal end section of the ultrasound image system used in the aforementioned step.

FIG. 24 illustrates a step of the catheter ablation.

FIG. 25 illustrates an ultrasound image obtained by the ultrasound image system used in the aforementioned treatments.

FIG. 26 illustrates an ultrasound image obtained by the ultrasound image system used in the aforementioned treatments.

FIG. 27 illustrates a step of the catheter ablation.

FIG. 28A is an enlarged view showing a part of a distal end section of the ultrasound image system according to a fourth embodiment of the present invention. FIG. 28B shows a deformed state of the distal end section.

FIG. 29 shows bow to indwell a stent graft by using the ultrasound image system.

FIG. 30 shows a retained state of the stent graft by using the ultrasound image system.

### DETAILED DESCRIPTION OF THE INVENTION

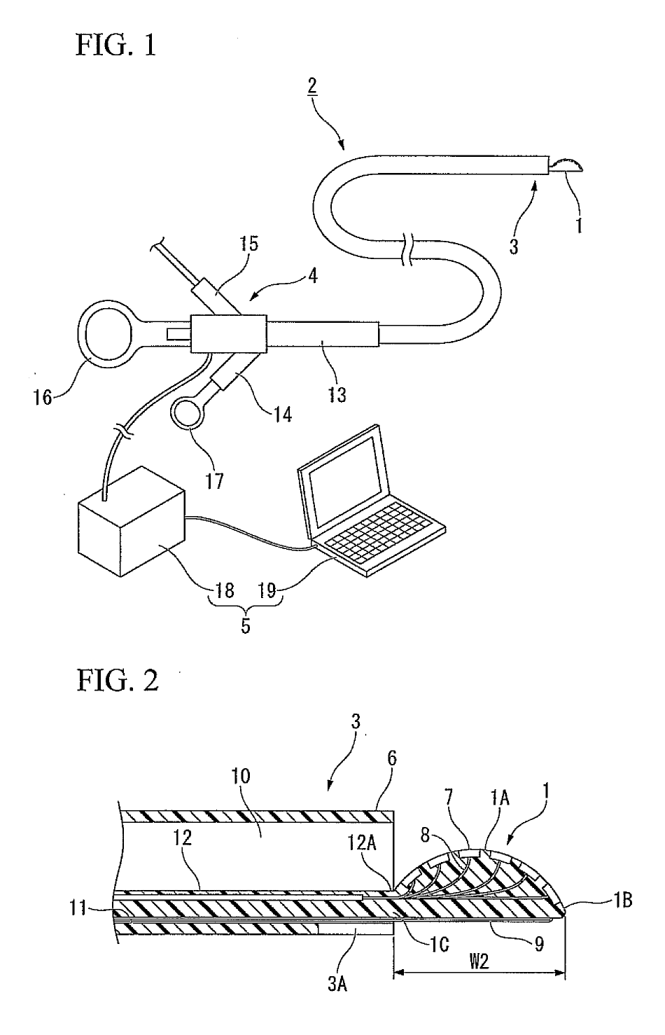

A cardiovascular ultrasound probe (hereinafter simply called a “probe”) and an ultrasound image system according to a first embodiment of the present invention are explained as follows with reference to FIGS. 1 to 10.

FIG. 1 is a schematic view of an ultrasound image system 2 provided with a probe 1 according to the present embodiment. The ultrasound image system 2 is provided with: a tip section 3 having the probe 1 thereon; a maneuvering section 4 provided to the proximal end of the probe 1; and an image-processing section 5 that conducts image-processing and image-display with respect to a signal obtained by the probe 1.

FIG. 2 is an enlarged view showing a part of a tip section 3 in cross-section. The tip section 3 is provided with the probe 1 and a sheath 6 having the probe 1 at a distal end thereof.

The probe 1, which is made of an elastic material, e.g., resin, has a convex surface 1A for transmitting and receiving ultrasound waves. A plurality of ultrasound transducers 7 (hereinafter simply called “transducers”) is disposed on the transmitting-and-receiving surface 1A. The plurality of transducers 7 is disposed at an interval of approximately 0.2 mm along the transmitting-and-receiving surface 1A. In addition, a width W2 of the probe 1 is approximately 3 mm by curving the transmitting-and-receiving surface 1A.

The frequency of ultrasound wave transmitted by the transducers 7 should be preferably in a range of 20 to 40 megahertz (MHz) in view of required resolution, and the like. Cables 8 are connected to each transducer 7 that receives reflected ultrasound wave and transmits the corresponding signal to an image-processing section 5.

A maneuvering wire 9 for changing the direction of the transmitting-and-receiving surface 1A is connected to an end section 1B provided at the distal end of the probe 1. The proximal end of the maneuvering wire 9 is inserted through a second lumen of the sheath 6 that will be explained later.

The sheath 6 is a tubular member made of an elastic material, e.g., resin. The sheath 6 has two lumens, i.e.: a first lumen 10 that allows various instruments including a guidewire to be inserted therethrough; and a second lumen 11 through which the maneuvering wire 9 is inserted. The first lumen 10 and the second lumen 11 are separated by a septum 12.

An end section 1C provided to the proximal end of the probe 1 is connected to a distal end 12A of the septum 12 so that the transmitting-and-receiving surface 1A is directed inward with respect to a radial direction of the sheath 6. The cables 8 passing through the septum 12 are connected to the image-processing section 5.

The maneuvering wire 9 capable of extending and retracting in an axial direction is inserted through the second lumen 11. Provided on a wall surface of the sheath 6 in the vicinity of the second lumen 11 is a slit 3A that allows the maneuvering wire 9 to come out of the sheath 6 when maneuvering the probe 1.

As illustrated in FIG. 1, the maneuvering section 4 is provided with a main body 13 having the proximal end of the sheath 6 fixed thereto; a wire-maneuvering section 14 for maneuvering a maneuvering wire; and an insertion section 15 through which an instrument, and the like. is inserted.

The main body 13 is a substantial cylindrical member having two lumens that are not shown in the drawings. The proximal end of the sheath 6 is fixed to the distal end of the main body 13 by a bonding method or welding method. The two lumens each communicate with the first lumen 10 and the second lumen 11 of the sheath 6. Provided at the proximal end of the main body 13 is a handle 16 for manipulating the rotation.

A wire-maneuvering section 14 is provided to the main body 13 so that the wire-maneuvering section 14 communicates to The second lumen 11. The proximal end of the maneuvering wire 9 is exposed from the wire-maneuvering section 14. A wire handle 17 for manipulating extension and retraction of the maneuvering wire 9 is attached is attached to the proximal end of the maneuvering wire 9.

The insertion section 15 is provided to the main body 13 so that the insertion section 15 communicates to the first lumen 10. Various instruments and apparatuses are inserted into the insertion section 15.

An image-processing section 5 is provided with a processor 18 having the cable 8 connected thereto; and a personal computer 19 connected to the processor 18.

Signals including reflected ultrasound waves received by the transducer 7 of the probe 1 via the cable 8 are transmitted to the processor 18. The signals undergo common wave-processing based on stored programs or on user's instructions input via into the personal computer 19; thus, the processed signals are reconfigured to become ultrasound images. A variety of information including the ultrasound images transmitted from the processor 18 is displayed on a display section of the personal computer 19.

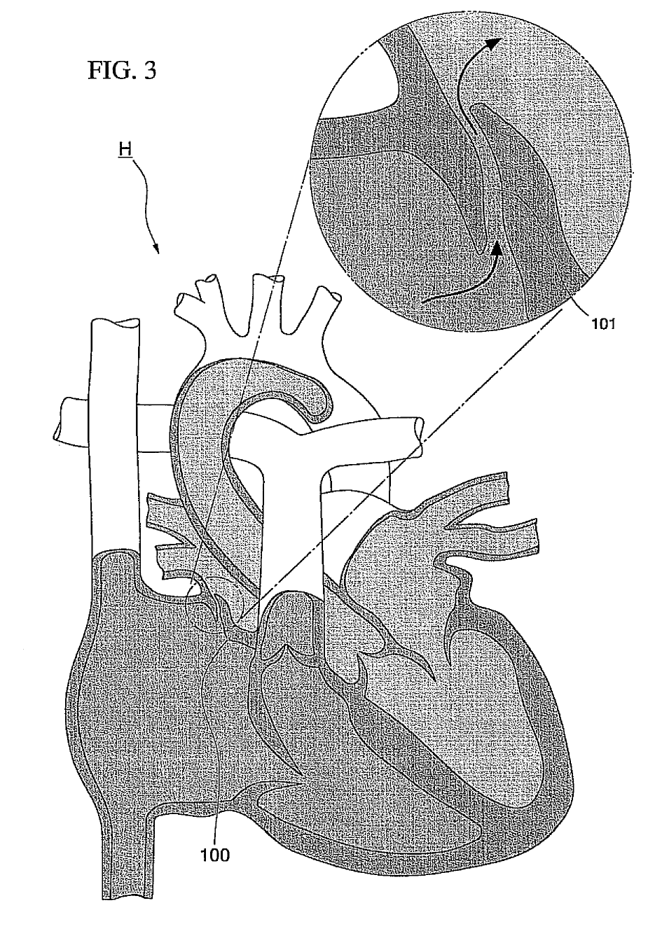

Operations using the ultrasound image system 2 having the previously explained configuration will be explained. An example of a treatment using the ultrasound image system 2 is obturation to a patent foramen ovale (hereinafter called “PFO”) 101 existing in an atrial septum 100 of a heart H as illustrated in FIG. 3.

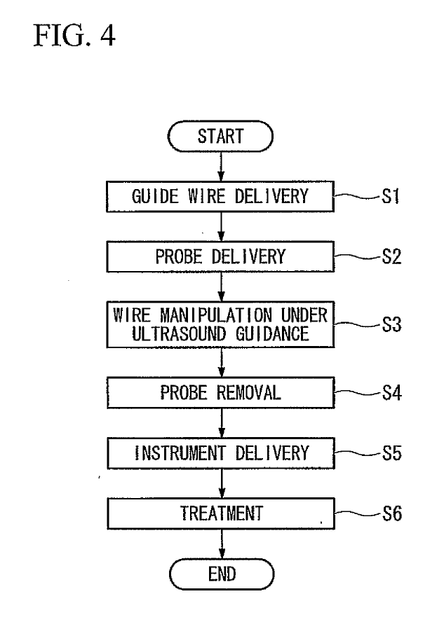

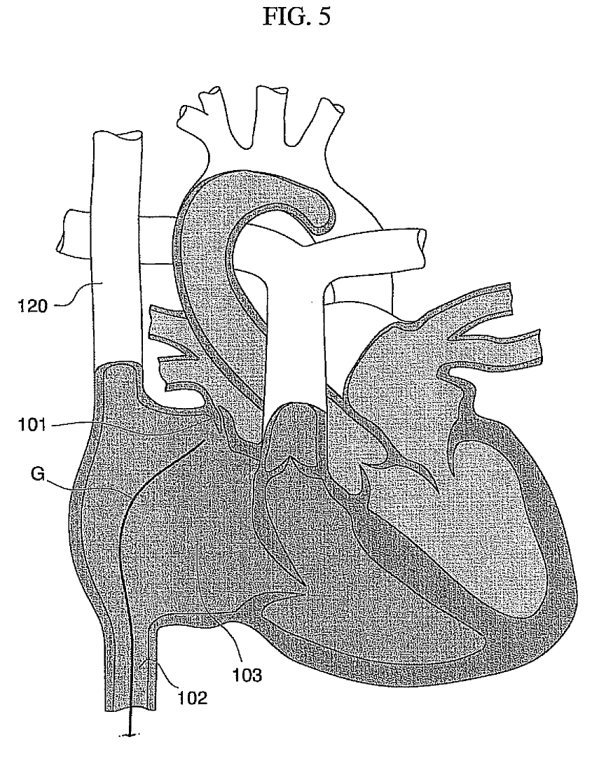

FIG. 4 is a flowchart showing a sequence of the treatment. To begin with, in Step S1, a user punctuates and inserts a guidewire (instrument) G into a femoral vein of a patient. Consequently, as illustrated in FIG. 5, the guidewire G is further fed from a inferori vena cava 102 to the right atrium 103 so that the distal end of the guidewire G is conveyed into the right atrium 103. It should be noted that the right atrium 103 may be approached from a superior caval vein, and in that case, a cervical vein or a vein of a brachium is punctuated.

Commonly, the above-explained delivery of the guidewire G is conducted under observation using a chest X-ray Fluoroscopy image (hereinafter simply called “X-ray image”). Although X-ray image may suffice to move the distal end of the guidewire G to the same height as that of the PFO 102, maneuvering the distal end of the guidewire G to a target, i.e., the PFO 101 accurately requires skill since the information associated with anteroposterior direction in the patient cannot obtained from the X-ray image.

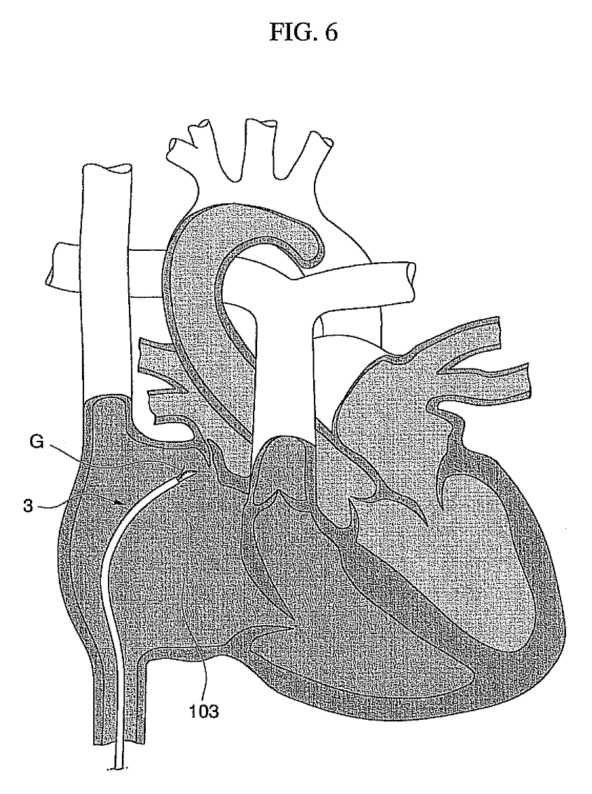

In the following Step S2, the user inserts the proximal end of the guidewire G into the first lumen 10 from the probe 1. Consequently, a tip section 3 is moved along the guidewire G into the right atrium 103 as illustrated in FIG. 6.

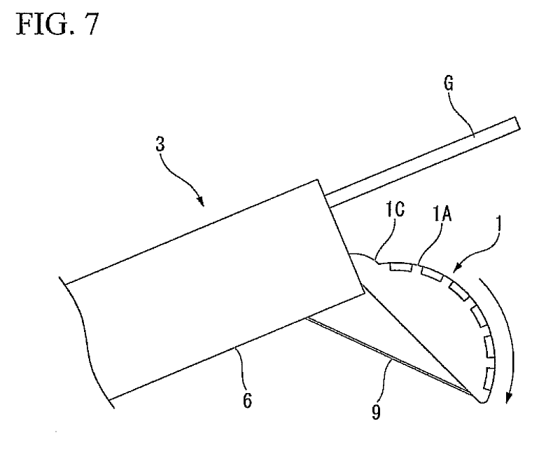

Upon observing the probe 1 positioned in the right atrium 103, the user retracts the wire handle 17 to retract the maneuvering wire 9 appropriately. Then, as illustrated in FIG. 7, the probe 1 rotates outward in radial direction of the sheath 6 around a center, i.e., an end section 1C connected to the sheath 6. Accordingly, a transmitting-and-receiving surface 1A of the probe 1 moves to a position that allows ultrasound waves to be transmitted in a substantial front of the sheath 6 including the distal end of the guidewire G projecting from the first lumen 10.

Having maneuvered the rotation of the probe 1, the user in Step S3 turns on a power supply that is not shown in the drawing and actuates a transducer 7 on the probe 1 to transmit ultrasound wave from the transmitting-and-receiving surface 1A. The reflected waves received by the transducer 7 are transmitted to the processor 18 through the cable 8 and is processed there; thus, ultrasound image obtained by the probe 1 is displayed on the personal computer 19. The user pauses the X-ray image when the distal end of the guidewire G is at the same height of that of the PFO 101, and moves the distal end of the guidewire G to the PFO 101 based on the ultrasound image obtained by the probe 1. It should be noted that guidance using the X-ray image may be continued if necessary.

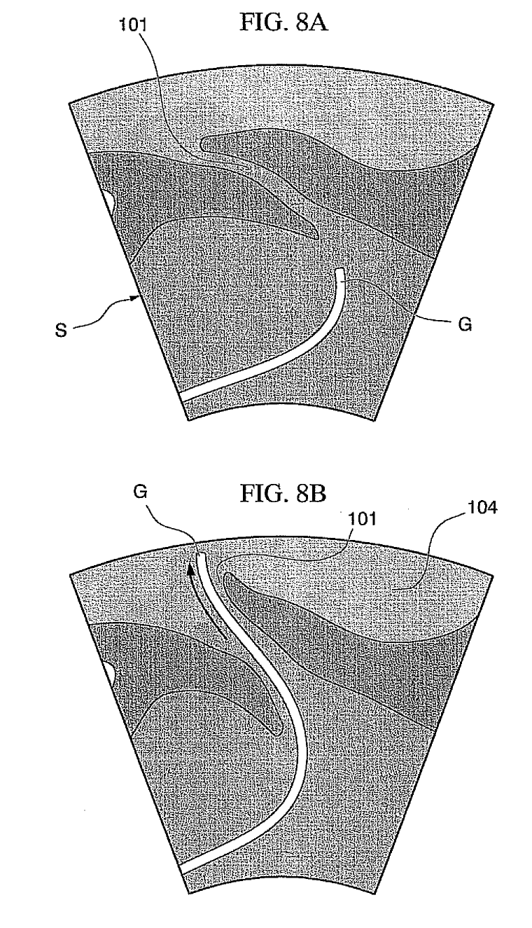

FIGS. 8A and 8B show an ultrasound image obtained by the probe 1. A perspective S is in a substantial fan shape since ultrasound wave is transmitted in a fan shape from the transducers 7 disposed on the transmitting-and-receiving surface 1A. The correlation between the probe 1 and the first lumen 10 is continuously maintained since the probe 1 is connected to the sheath 6. As illustrated in FIG. 8A, the distal end of the guidewire G projecting from the first lumen 10 is captured in the perspective S. A position to start describing the guidewire G can be changed in accordance with feeding amount of the maneuvering wire 9. It should be noted that ultrasound images in the following explanations are schematic, that is, the images may look somewhat different upon visual inspection.

In a case where the PFO 101 or the distal end of the guidewire G cannot be captured desirably, fine control may be conducted appropriately, e.g., the transmitting-and-receiving surface 1A of the probe 1 is rotated around the axial line of the sheath 6 by rotating the main body 13 and the sheath 6 by using the handle 16.

The user inserts the guidewire G into the PFO 101 as illustrated in FIG. 8B by micro-controlling the distal end of the guidewire G with respect to the anteroposterior direction lateral direction in FIG. 8A) while adjusting the correlation relative to the PFO 101.

Having observed that the guidewire G is inserted through the PFO 101 and the distal end projects into the left atrium 104, the user turns off the probe 1 and removes the tip section 3 in Step S4.

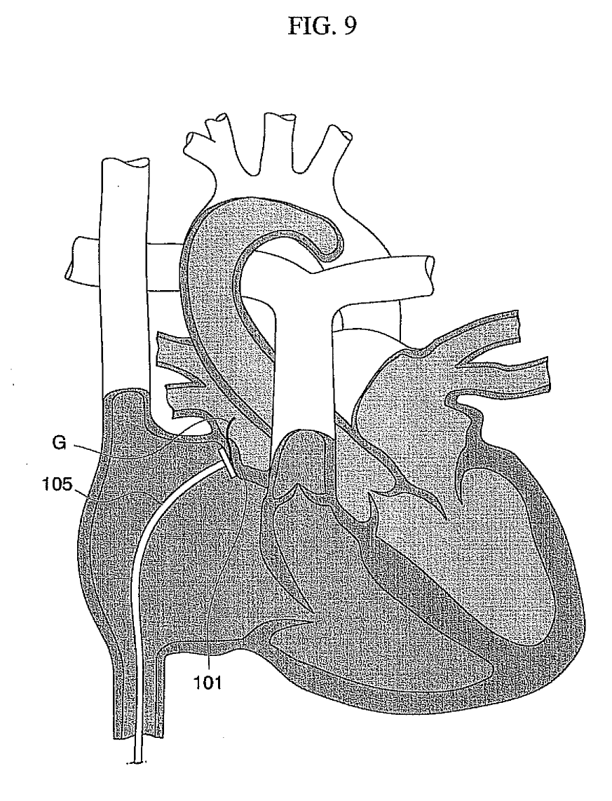

In Step S5, the user moves the instrument 105 to the PFO 101 along the guidewire G under guidance of X-ray image for a second time as illustrated in FIG. 9. The instrument 105 used here is a common instrument for obturating a PFO as disclosed by U.S. Publication No. 2006-0271030. Since the guidewire G has been inserted into the PFO 101 previously, this state of distal end of the instrument 105 along the guidewire G will be advanced and reach the PFO 101 smoothly.

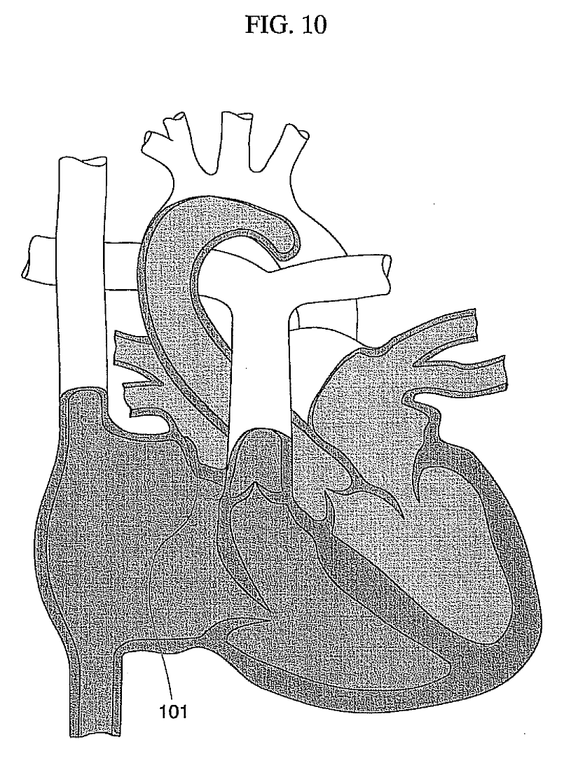

In Step S6, the user presses the distal end of the instrument 105 onto the PFO 101 while supplying electric current there; thus, the PFO 101 is adheded, i.e., obturated, as illustrated in FIG. 10. After the treatments, the user removes the instrument 105 and the guidewire G; thus, a sequence of the manipulations is completed.

The probe 1 of the ultrasound image system 2 according to the present embodiment can obtain an ultrasound image substantially in front of the guidewire G inserted through the first lumen 10 of the sheath 6. Although X-ray image cannot obtain the information about the patient with respect to the position of the target, i.e., the PFO 101 in the anteroposterior direction, it can be obtained in the present embodiment. Therefore, the guidewire G can be inserted and reach the PFO 101 more easily.

The dimension of the tip section 3 being inserted can be restricted to the extent that is equivalent to that of a conventional ultrasound probe used in blood vessels since the end section 1C of the probe 1 is connected to the distal end of the sheath 6; and the transmitting-and-receiving surface 1A can be directed substantially forward by retracting the maneuvering wire 9. Accordingly, a probe having more significant insertability can be configured.

Furthermore, the guidewire G and the probe 1 inserted through the first lumen 10 can be maneuvered in substantially one unit since the correlation between the probe 1 and the sheath 6 can be maintained in a fixed state. Therefore, the delivery of the guidewire G can be facilitated while reliably obtaining a view in front of the distal end of the ultrasound image guidewire G since the distal end of the guidewire G can be captured in the perspective S of the ultrasound image captured by the probe 1.

The present invention is not limited to the example where the guidewire G according to the present embodiment is introduced by using the probe 1. For example, in a case where an instrument 105 for treating a PF0 is of a type that is difficult to be fixed to the optimum position relative to the PFO, the instrument may be introduced and fixed based on an ultrasound image obtained by the probe 1 while inserting the instrument through the first lumen 10. The ultrasound image in this case of the probe 1 can introduce and fix the instrument desirably and enables to observe appropriate treatment on an ultrasound image.

Also, an object of the guidewire introduction, i.e., disease is not limited to PFO. For example, the guidewire introduction may be used when a lead electrode is inserted through a coronary sinu.

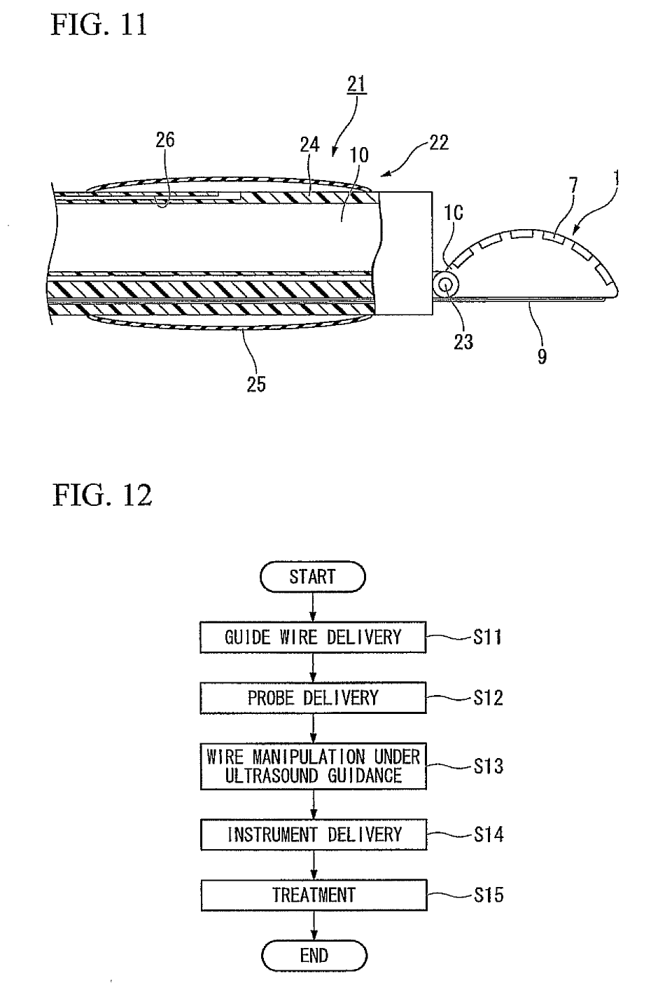

A second embodiment of the present invention will be explained next with reference to FIGS. 11 to 19. An ultrasound image system 21 according to the present embodiment is different from the above-explained ultrasound image system 2 due to the different connection between a probe and a sheath and due to a balloon attached to the sheath.

Note that elements that are equivalent to the ultrasound image system 2 according to the first embodiment will be assigned the same reference symbols and redundant explanations thereof will be omitted in each of the following embodiments.

FIG. 1 is an enlarged view showing a part of a tip section 22 of the ultrasound image system 21 in cross-section. An end section 1C of a probe 1 that is identical to that of the first embodiment is connected to a sheath 24 via a hinge 23.

A balloon 25 made of a resin, e.g., polyethylene terephthalate (PET) or a rubber is provided to an outer periphery in the vicinity of the distal end of the sheath 24. An inflation tube 26, expanding close to the proximal end of the sheath 24, for inflating the balloon 25 is provided on a wall surface of the sheath 24. The inflation tube 26 is connected to an inflator, not shown in the drawings, at the proximal end of the sheath 24. It should be noted that the inflation tube 26 may be configured to penetrate the wall surface of the sheath 24 and to expand to the proximal end of the sheath 24 along the first lumen 10.

Operations for treating a chronic total occlusion (hereinafter called “CTO”) of an arteria coronaria by using the above-configured ultrasound image system 21 are explained with reference to FIGS. 12 to 19.

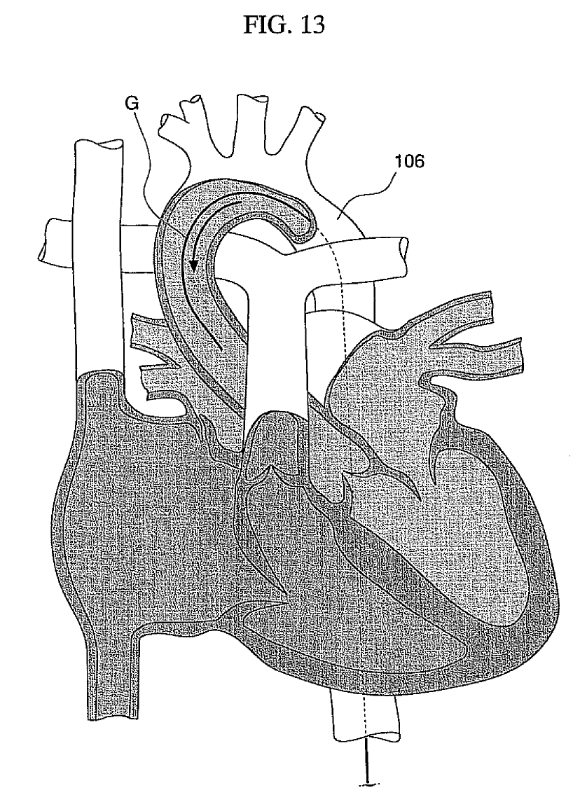

FIG. 12 is a flowchart showing a sequence of the treatment. To begin with, in Step S11, a user punctuates a femoral artery of a patient and conveys a guidewire G to a starting point of an aorta 106 under X-ray image observation as illustrated in FIG. 13. Consequently, the guidewire G ingresses into an arteria coronaria where a target disease, i.e., a CTO, exists. The aorta 106 may be approached from a carotid artery or a brachial artery instead of the femoral artery.

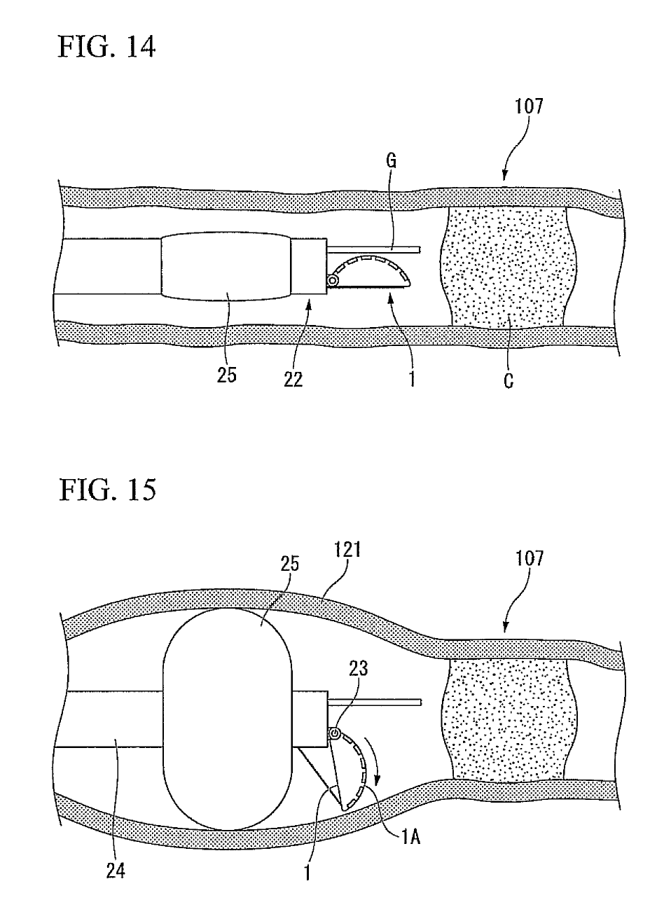

Consequently, in the Step S12, the user moves the tip section 22 in the vicinity of the CTO 107 along the guidewire G as illustrated in FIG. 14 by the step equivalent to the Step S2 of the first embodiment. The proximal end of the guidewire G is projected from an insertion section 15 that is not shown in the drawings.

A contrast agent does not flow distally relative to a CTO 107 because blockagean obstruction C made of a clot, etc., fully blocks the arteria coronaria. Therefore, although a distal portion in a running direction of blood relative to the CTO 107 cannot be observed on X-ray image. FIG. 14 illustrates a distal portion relative to the CTO 107. An attempt to penetrate the blockage C by the guidewire G, etc., to penetrate this state of CTO may threaten to break through a wall of blood vessel depending on the running direction of blood vessel distal relative to the blockage C.

The treatments proceeds to Step S13 wherein the guidewire G is maneuvered by using the probe 1 of the tip section 22 under sonography. To begin with, the user supplies air or normal saline solution into the balloon 25 via an inflation tube 26, not shown in the drawings, by maneuvering the inflator as illustrated in FIG. 15. Accordingly, the arteria coronaria 121 is expanded and a space for rotating the probe 1 therein is provided.

This state of arteria coronaria 121 blocked by the balloon 25 is not a significant problem since the blocking lasts for a short time and the arteria coronaria 121 has been blocked previously.

Upon expanding the arteria coronaria 121, the user retracts the maneuvering wire 9. After a while, the probe 1 rotates outward in radial direction of the sheath 24 around a center, i.e., hinge 23; thus, a transmitting-and-receiving surface 1A is directed to the CTO 107. The user pauses the X-ray image temporarily, and switches to an ultrasound image obtained by the probe 1.

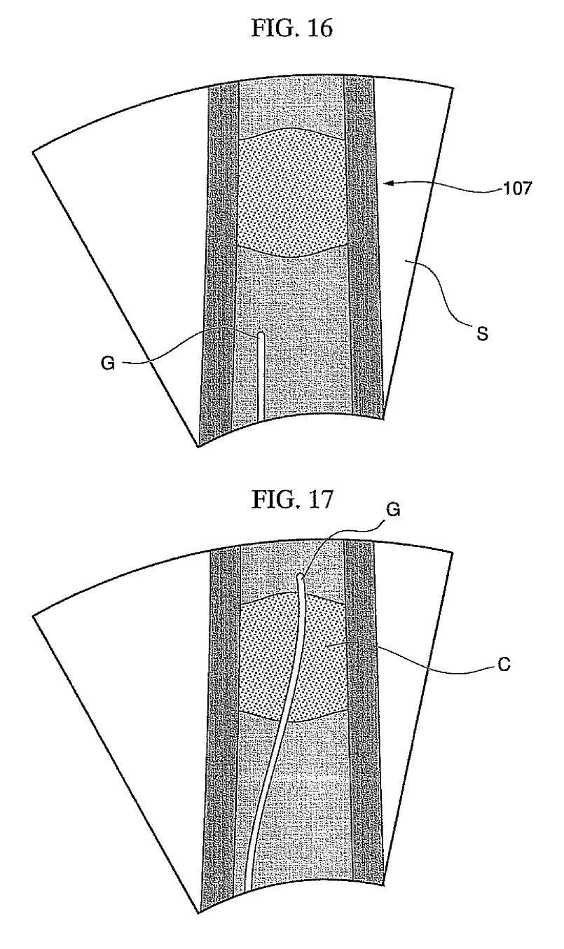

FIGS. 16 to 19 show an ultrasound image obtained by the probe 1. The distal end of the guidewire G is captured in a perspective S as illustrated in FIG. 16. In addition, a wall of a blood vessel that is distal relative to the CTO 107 is described. The user maneuvers the guidewire G and penetrates the blockage C while observing the running direction of the distal blood vessel as illustrated in FIG. 17.

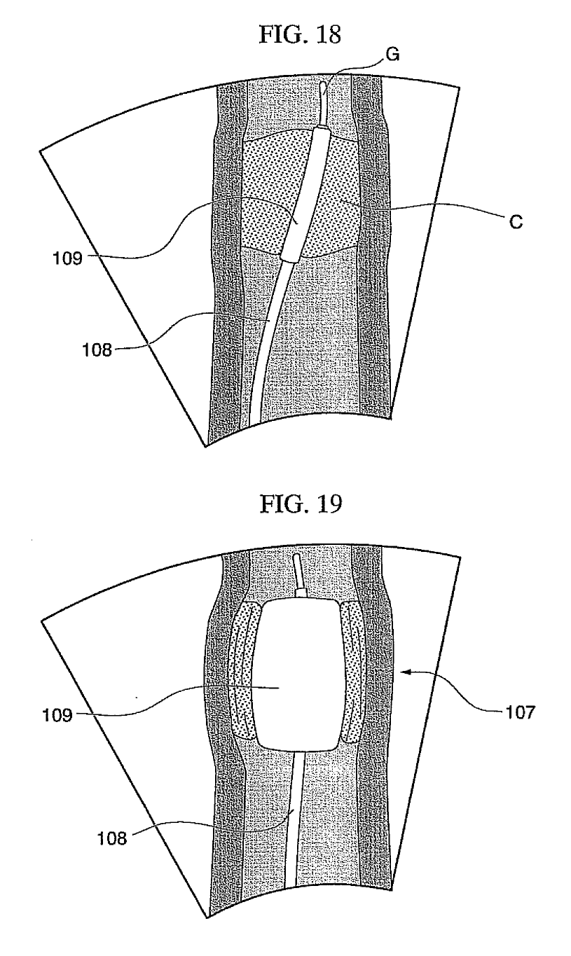

Consequently, in Step S14, the user inserts a balloon catheter (instrument) 108 into a first lumen 10 from an insertion section 15, not shown in the drawings, and passes the balloon catheter 108 along the guidewire G through the blockage C as illustrated in FIG. 18. Preferably, the balloon 108 of the balloon catheter 108 should be selected that has a length in an axial line direction that is equivalent to that of the blockage C in an axial direction.

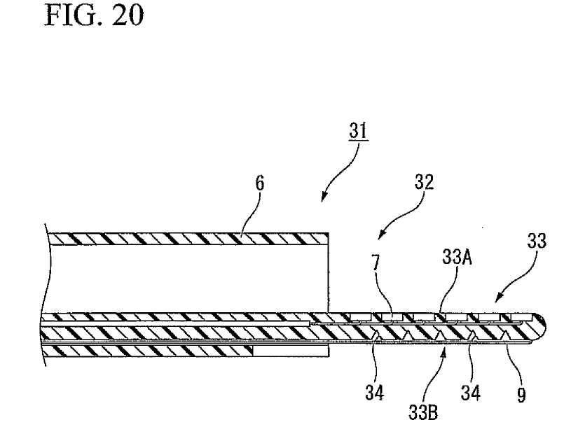

As illustrated in FIG. 19, the user inflates the balloon 109 by using the inflator, not shown in the drawings, connected to the balloon catheter 108, and penetrates through the CTO 107 while observing an ultrasound image captured by the probe 1. Consequently, the sequence of treatment is completed after removing the instruments, switching to X-ray image, and inspecting the completion of the penetration by causing a contrast agent to flow.

The probe 1 in the maneuvering wire 9 according to the present embodiment rotates more smoothly by proximally retracting the maneuvering wire 9 since the probe 1 is connected to the sheath 24 via the hinge 23. Therefore, the transmitting-and-receiving surface 1A can be directed forward more desirably while maintaining the correlation to the sheath 24. Therefore, the condition in front of the disease like CTO 107 can be observed reliably, and it can be observed by an ultrasound image that a safe and accurate treatment has been conducted without causing damage to the wall of the blood vessel.

Also, a space for rotating the probe 1 can be obtained in a relatively narrow area such as an arteria coronaria 121 by expanding the balloon 25 since the balloon 25 is provided in the vicinity of the distal end of the sheath 24.

A third embodiment of the present invention will be explained next with reference to FIGS. 20 to 27. An ultrasound image system 31 according to the present embodiment is different from the above-explained ultrasound image system 2 with respect to the structure of a probe.

Note that elements that are equivalent to the ultrasound image system 2 according to the first embodiment will be assigned the same reference symbols and redundant explanations thereof will be omitted in each of the following embodiments.

FIG. 20 is an enlarged view showing a part of a tip section 32 of the ultrasound image system 31 in cross-section. A substantially bar-shaped probe 33 made of a flexible material has a transmitting-and-receiving surface 33A having transducers 7 similar to those of the first embodiment disposed at a constant pitch. A plurality of identical grooves 34, having a substantial V-letter-shaped cross section, is formed on the transmitting-and-receiving surface 33A and on the opposite deformable surface 33B. The grooves 34 extend in a depth direction with respect to FIG. 20. As explained later, each groove 34 serves for deforming the probe 33 desirably. Deformation will provide a convex-shaped ultrasound transducer that is capable of ultrasound diagnosis in front thereof.

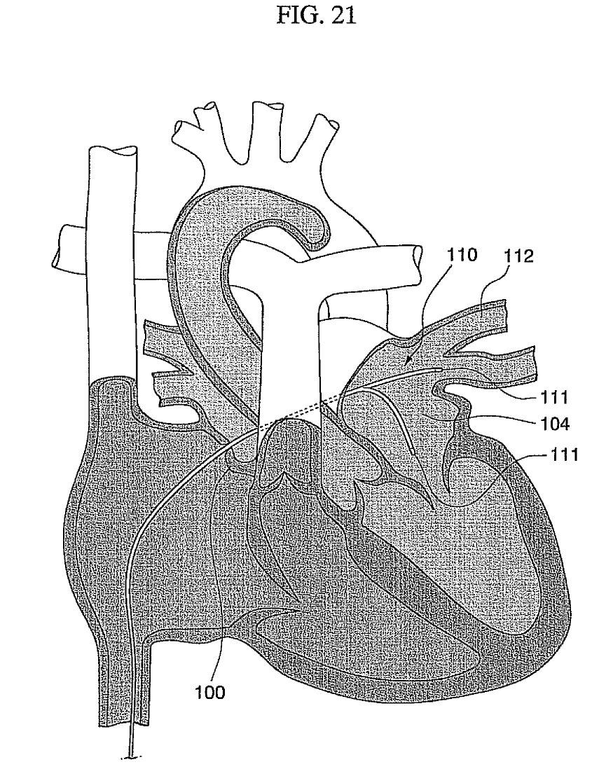

An example with reference to FIGS. 21 to 27 is a catheter ablation using the above-configured ultrasound image system 31 for curing an atrial fibrillation.

To begin with, as a preparatory step, remedial mapping is conducted wherein an electrocardiogram electrode 111 provided at the distal end of the catheter 110 having penetrated an atrial septum 100 is disposed in an left atrium 104 to search for a cauterization target such as an abnormal conducting path or a hypersthenic node, etc. The following explanation is based on an assumption based on the above mapping indicating that the cauterization target exists in the vicinity of an opening of a blood vessel 112 (hereinafter called “pulmonary vein”) that is one of pulmonary veins running into the left atrium 104.

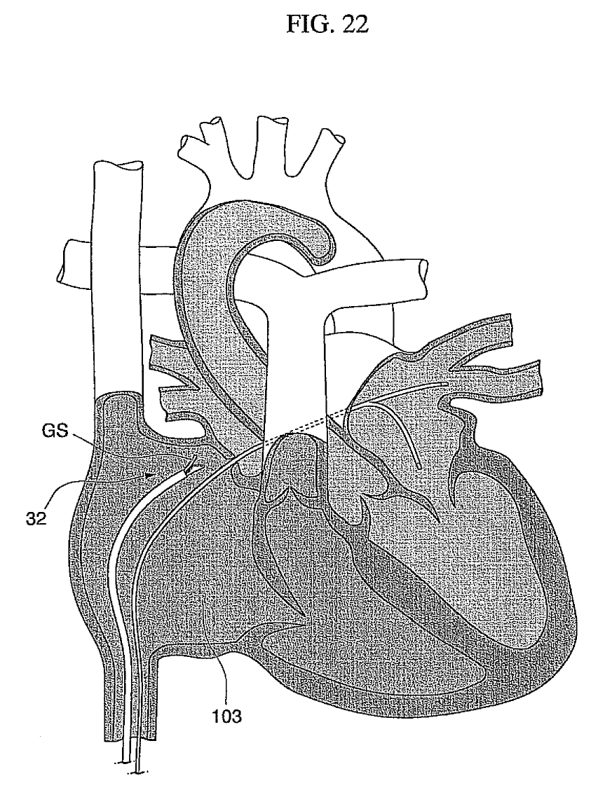

The user first conveys a tubular guide sheath (instrument) GS into an right atrium 103 by a method used for the guidewire G according to the first embodiment and consequently moves the tip section 32 along the guide sheath GS into the right atrium 103 as illustrated in FIG. 22.

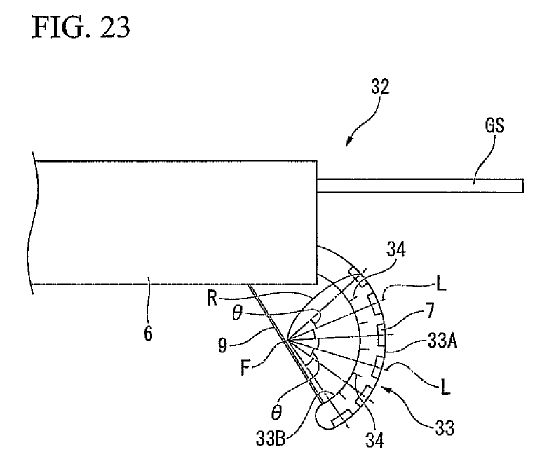

The user retracts the maneuvering wire 9 in a proximal direction. Then, as illustrated in FIG. 23, the grooves 34 formed on the deformable surface 33B of the probe 33 stop up while deforming into a substantial bow-shape; thus the probe 33 rotates outward in a radial direction of the sheath 6. This state of transducers 7 provided on the transmitting-and-receiving surface 33A make a substantial arc shape while the shape of groove 34 is configured so that normal L crossing each transducer 7 with respect to the transmitting-and-receiving surface 33A passes through a center point F, and so that an angle θ defined by adjacent normals is identical.

Accordingly, maneuvering of the maneuvering wire 9 imparts a convex shape defined by the interval and shape of the grooves 34. The transducers 7 are arranged on the transmitting-and-receiving surface 33A with a curvature R and the angle pitch θ. Simultaneously charging each transducer 7 with voltages having different phases from a power supply, not shown in the drawings, causes an ultrasound beam in front of the guide sheath GS. An ultrasound image in front of the guide sheath GS is produced by sequentially switching the transducers.

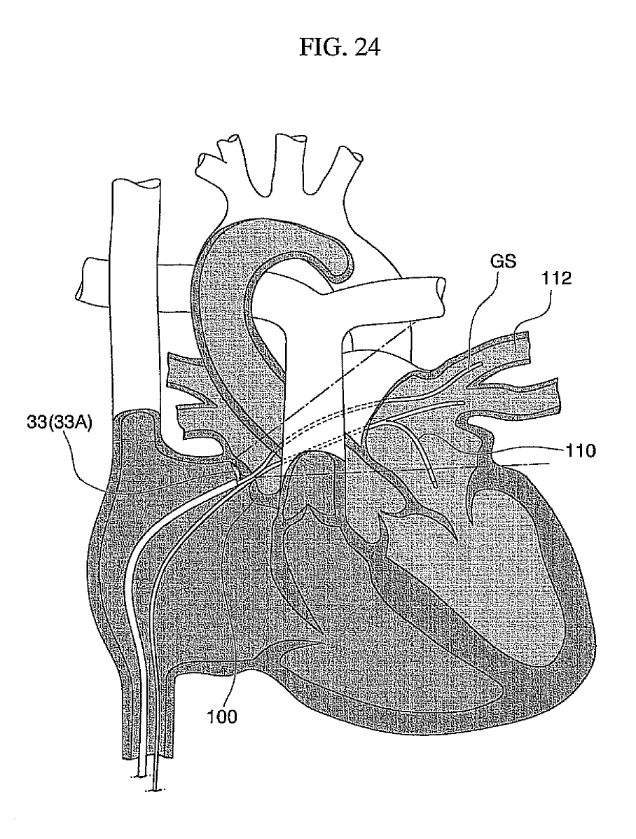

As illustrated in FIG. 24, the user inserts only the guide sheath GS through a hole on an atrial septum 100 that has a catheter 110 inserted therethrough, and presses the transmitting-and-receiving surface 33A of the probe 33 onto the atrial septum 100 while fixing the maneuvering wire 9 by means of a clip, etc., and maintaining the deformed shape of the probe 33. The user pauses the X-ray image and switches to an ultrasound image obtained by the probe 33. Consequently, the user moves the distal end of the guide sheath GS to the vicinity of the pulmonary vein while observing the left atrium across the atrial septum 100.

It should be noted that the guide sheath GS may be passed through the atrial septum 100 under guidance of ultrasound image obtained by the probe 33.

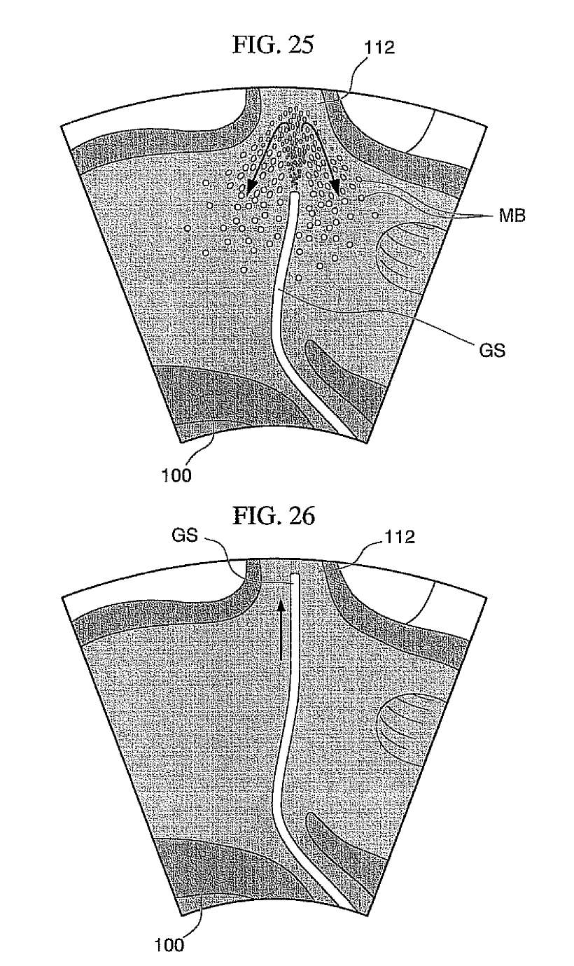

FIGS. 25 and 26 show an ultrasound image obtained by the probe 33 pressed onto the atrial septum 100. As illustrated in FIG. 25, the user causes contrast agent made of micro bubbles (Micro Bubble) MB to flow from the distal end of the guide sheath GS in order to inspect as to whether the distal end of the guide sheath GS faces the opening of the pulmonary vein. The microbubbles MB undergoing ultrasound resonation or burst to create a high brightness signal that can be observed on an ultrasound image.

The microbubbles MB are brought back by blood flowing from the pulmonary vein if the opening of the pulmonary vein faces the distal end of the guide sheath GS as illustrated in FIG. 25. The user observing the dynamics of the microbubbles MB on the ultrasound image can ascertain the correlation between the opening of the pulmonary vein and the distal end of the guide sheath GS.

Upon causing the distal end of the guide sheath GS to face the opening of the pulmonary vein, the user observing the position of the distal end of the guide sheath GS on the ultrasound image ingresses the guide sheath GS into the opening of the pulmonary vein as illustrated in FIG. 26.

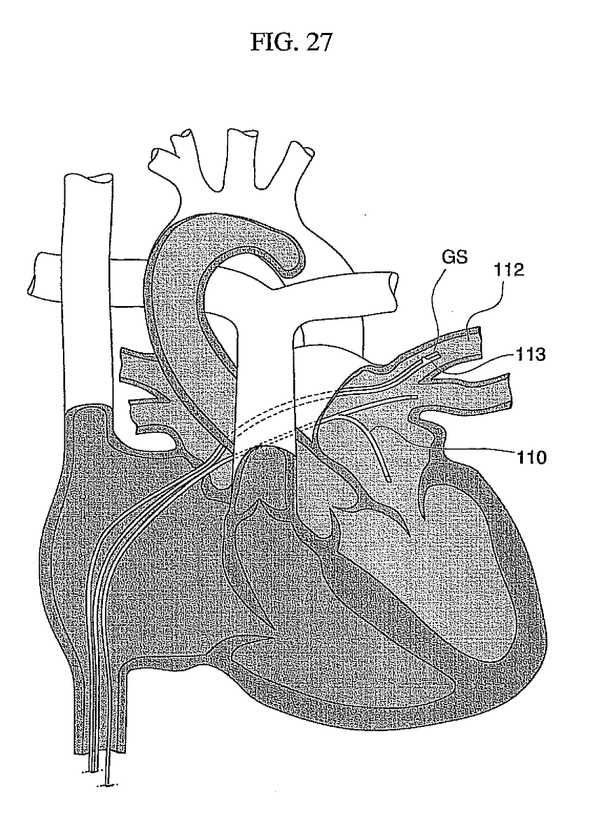

Upon ingressing the guide sheath GS into the pulmonary vein, the user removes the tip section 32 while the guide sheath GS is in place. Consequently, the user conveys an ablation catheter (instrument) 113 along the guide sheath GS to the pulmonary vein, and then completes the manipulations after cauterizing the cauterization target as illustrated in FIG. 27.

The substantial bar-shaped undeformed probe 33 of the ultrasound image system 31 according to the present embodiment can facilitate the insertion of the probe 33.

Instead of the example of the present embodiment wherein the position of the distal end of the guide sheath is observed by using contrast agent including microbubbles, the distal end may be controlled by attaching an ultrasound wave oscillator to the distal end of the guide sheath or a guidewire and observing the position of the oscillator on an ultrasound image obtained by the probe 33.

Alternatively, manipulative ablation under ultrasound observation may be conducted while a guide sheath and an ablation catheter are inserted through a first lumen without removing the tip section 32.

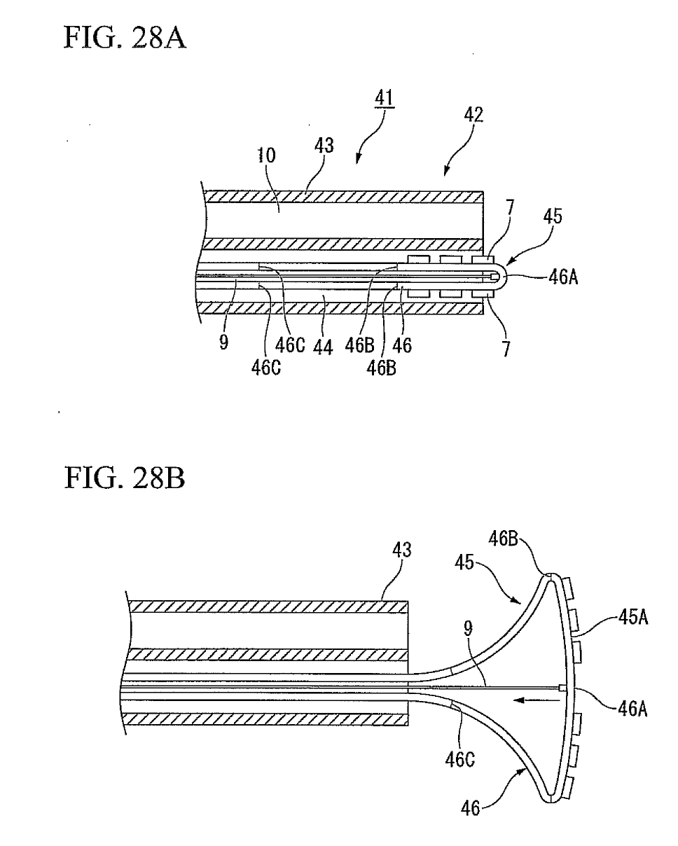

A fourth embodiment of the present invention will be explained next with reference to FIGS. 28A to 30. An ultrasound image system 41 according to the present embodiment is different from the above-explained ultrasound image system 2 in the structure of a probe.

Note that elements that are equivalent to the ultrasound image system 2 according to the first embodiment will be assigned the same reference symbols and redundant explanations thereof will be omitted in each of the following embodiments.

FIG. 28A is an enlarged view showing a part of a tip section 42 of the ultrasound image system 41 in cross-section. A sheath 43 is provided with two lumens, i.e., a first lumen 10 and a second lumen 44 housing therein a folded probe 45 capable of extending and retracting in an axial direction.

The probe 45 is provided with: a wire member 46; a transducers 7 that are identical to those of the first embodiment; and a maneuvering wire 9. The wire member 46 is folded at an intermediate section 46A having the transducers 7 on both sides of the intermediate section 46A at a constant pitch. Cables 8 (not shown in the drawings) passing inside of the wire member 46 are connected to the image-processing section 5. As explained below, creases 46B and 46C for deforming the probe 45 are formed at predetermined positions distal relative to the transducers 7 of the wire member 46.

The maneuvering wire 9 is connected to the intermediate section 46A of the wire member 46. The proximal ends of the maneuvering wire 9 and wire member 46 projecting from the wire-maneuvering section 14 enable the user to maneuver thereof.

The user, upon observing the distal end of the probe 45 projecting from the sheath 43 under X-ray fluoroscopy, fixes the outer periphery of the probe 45 to the sheath 43 by a probe stopper, not shown in the drawings, and retracts the maneuvering wire 9 proximally. Then the intermediate section 46A of the wire member 46 is retracted; thus, the wire member 46 bends at the crease 47B and 47C as illustrated in FIG. 28B. Accordingly, the deformed probe 45 forms a transmitting-and-receiving surface 45A directed to the front of the sheath 43. It should be noted that the wire member 46 may be deformable into other shape, e.g., sphere, and an umbrella, etc.

Steps using the above-configured ultrasound image system 41 for indwelling a stent graft into an abdominal aortic aneurysm are explained as follows.

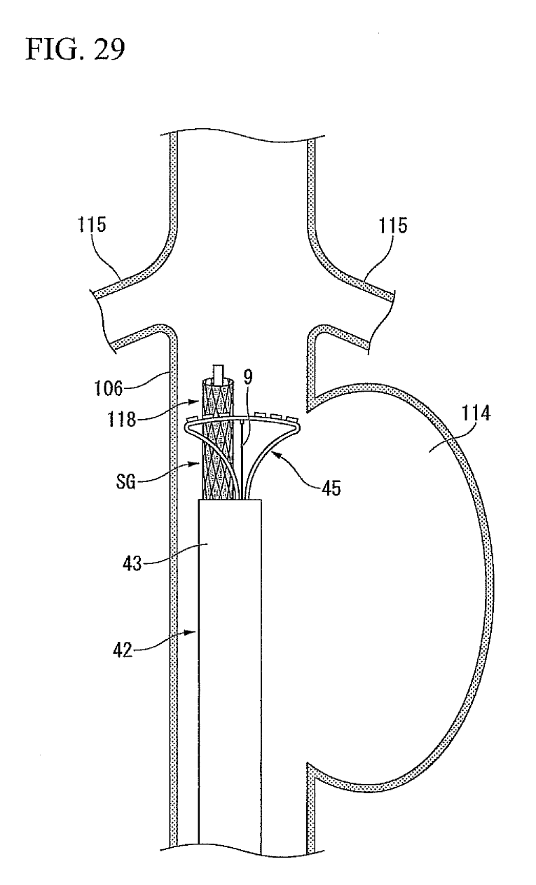

A user first punctuates a femoral artery of a patient and inserts a tip section 42 therein. Consequently, the user ingresses the tip section 42 to the vicinity of the abdominal aortic aneurysm (hereinafter called “AAA”) 114 of the aorta 106 while observing the position of the AAA 114 on an X-ray image by using contrast agent, etc. as illustrated in FIG. 29.

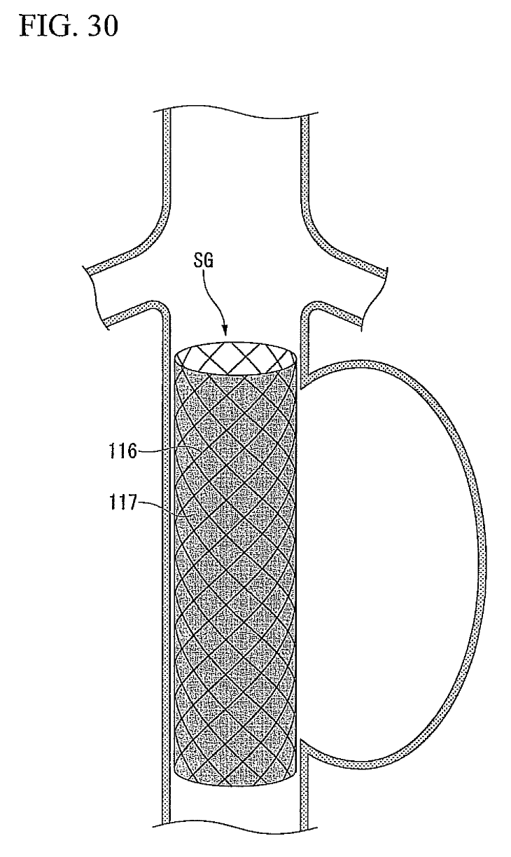

Many branch blood vessels 115 for supplying blood to various organs exist in the abdominal aorta. As illustrated in FIG. 30, a stent graft (instrument) SG has a support member 116 made from metal, etc. wherein the outer periphery of the support member 116 is surrounded by a coating 117. Therefore, the stent graft SG indwelled to overlap the branch blood vessels 115 may blocks blood flowing to the branch blood vessels 115; thereby causing serious complications.

In order to prevent this, the user ingresses the probe 45 to expose the probe 45 from the sheath 43 and retracts the maneuvering wire 9 to deform the probe 45. Consequently, the user switching to an ultrasound image and observing the position and destination of the branch blood vessels 115 determines a position where the stent graft SG is indwelled. In a case where the branch blood vessels 115 are hardly observed, the user manipulates a handle 16, not shown in the drawings, appropriately to rotate the probe 45.

Upon determining the position where the stent graft SG is indwelled, the user inserts the delivery system 118 of the stent graft SG into the first lumen 10 to expose the delivery system 118 from the distal end of the sheath 43 as illustrated in FIG. 29. Upon fixing the delivery system 118 and retracting the tip section 42, the support member 116 expands; thus the indwelling of the stent graft SG is completed as illustrated in FIG. 30. The AAA 114 is prevented from bursting since a precisely determined position for indwelling the stent graft SG based on the ultrasound image obtained by the probe 45 prevents the stent graft SG from blocking the branch blood vessels 115, and since blood supplied to the AAA 114 is blocked by the coating 117 of the stent graft SG.

The deformed probe 45 restores to the undeformed shape by fixing the outer periphery of the probe 45 to the sheath 43 by the probe stopper (not shown in the drawings) and pressing the maneuvering wire 9 in a distal direction; thus, the probe 45 can be housed in the sheath 43.

The undeformed probe 45 housed in the second lumen 44 of the sheath 43 can facilitate the smooth insertion of the tip section 42 in the ultrasound image system 41 according to the present embodiment.

In addition, more accurate indwelling of the stent graft SG can be conducted since more information, including a running direction of the branch blood vessels 115, than that obtained by a conventionally used intravascular ultrasound (IVUS) image is available because the deformed probe 45 captures an ultrasound image of ahead of the sheath 43 in a greater scope.

Although the present invention has been described with respect to its preferred embodiments, the present invention is not limited to the embodiments described above. The configuration of the present invention allows for addition, omission, substitution and further modification without departing from the spirit and scope of the present invention.

What is claimed is:

1. An ultrasound image system comprising: an ultrasound probe; a signal-transmitting-and-receiving section for transmitting signals to the ultrasound probe and receiving signals from the ultrasound probe; and an image-displaying section for displaying an ultrasound image obtained by means of the signal-transmitting-and-receiving section, wherein
the ultrasound probe comprises:
a tip section which is provided with: a plurality of ultrasound-wave-transmitting sections for transmitting ultrasound waves to an object tissue; and a plurality of ultrasound-wave-receiving sections for receiving signals reflected by the tissue and obtained by means of the ultrasound-wave-transmitting section; and
a flexible catheter tube provided with: a first lumen capable of inserting an installment for treating the tissue from a proximal end section of the ultrasound probe therethrough and exposing the instrument at a position facing the tip section; and a second lumen having an maneuvering wire inserted therethrough and being capable of moving the tip section.

2. The ultrasound image system according to claim 1, wherein the tip section is movable by maneuvering the maneuvering wire so that the ultrasound wave is transmitted toward the tissue disposed ahead in a longitudinal direction of a cable.

3. The ultrasound image system according to claim 2, wherein an instrument port for passing through a first tubular space and projecting the instrument from a distal end of the catheter tube is disposed in a position facing the tip section, and the instrument is disposed in an observation perspective obtained by means of the ultrasound probe.

4. The ultrasound image system according to claim 3, further comprising an inflation section, provided at least a part of the catheter tube, which is inflated by filling air or liquid thereinto to provide a space which allows the tip section to move.

5. The ultrasound image system according to claim 2, wherein the tip section can be retracted into the catheter tube.

6. The ultrasound image system according to claim 1, wherein the catheter tube is capable of being inserted into a blood vessel or a heart, and is capable of X-ray projection.

7. A method of observing or treating the inside of a blood vessel or the inside of a heart by using a ultrasound probe, an ultrasound image obtained by the ultrasound probe, and a instrument for treating an tissue, the method comprising:
a step of inserting the instrument into the ultrasound probe;
a step of moving a tip section of the ultrasound probe;
a step of adjusting a perspective of the ultrasound image; and
a step of guiding the instrument to a treatment section accurately based on the ultrasound image.

8. An ultrasound probe comprising:
a tip section which is provided with: a plurality of ultrasound-wave-transmitting sections for transmitting ultrasound wave to an tissue; and a plurality of ultrasound-wave-receiving sections for receiving signals reflected by the tissue and obtained by means of the ultrasound-wave-transmitting section; and
a flexible catheter tube provided with: a first lumen capable of inserting an instrument for treating the tissue from a proximal end section of the ultrasound probe and exposing the instrument at a position facing the tip section; and a second lumen having an maneuvering wire capable of moving the tip section inserted therethrough.
